(19) United States
(12) Patent Application Publication
(10) Pub. No.: US 2009/0197295 A1
(43) Pub. Date: Aug. 6, 2009

(54) MASKS USEFUL FOR MALDI IMAGING OF TISSUE SECTIONS, PROCESSES OF MANUFACTURE AND USES THEREOF

(76) Inventors: Isabelle Fournier, Bourghelles (FR); Vincent Thomy, Fretin (FR); Michel Salzet, Boughelles (FR); Maxence Wisztorski, Lille (FR); Nicolas Verplanck, Wattignies (FR)

Correspondence Address: FOLEY AND LARDNER LLP;SUITE 500, 3000 K STREET NW, WASHINGTON, DC, 20007, US

(21) Appl. No.: 12/298,868

(22) PCT Filed: May 2, 2007
(86) PCT No.: PCT/EP07/54253

§ 371 Date: Oct. 28, 2008

Related U.S. Application Data
(60) Provisional application No. 60/796,535, filed on May 2, 2006.

(51) Int. Cl. C12Q 1/02; H01J 49/00; B44C 1/22; B05D 5/12; H01J 5/18

(52) U.S. Cl. 435/29; 250/281; 216/17; 427/58; 250/505.1

(57) ABSTRACT

The present invention relates to masks for use in mass spectrometry, in particular MALDI, tissue section analysis, comprising a plate made of or coated by an opaque material and having a thickness of less than 150 μm, said plate comprising regularly spaced openings, wherein in the plate upper plane, the diameter D of the largest circle comprising only one opening is superior to the diameter d of a mass spectrometer, in particular a MALDI analyzer, laser beam divided by sin Θ, wherein Θ is the mass spectrometer, in particular a MALDI analyzer, laser beam incidence angle with respect to the sample plane. The invention also concerns processes of manufacture of the masks according to the invention, the use thereof for mass spectrometry, in particular MALDI, imaging of tissue sections, and a method for MALDI imaging of a tissue section using said masks.

The present invention relates to masks for use in mass spectrometry, in particular MALDI, tissue section analysis, comprising a plate made of or coated by an opaque material and having a thickness of less than 150 μm, said plate comprising regularly spaced openings, wherein in the plate upper plane, the diameter D of the largest circle comprising only one opening is superior to the diameter d of a mass spectrometer, in particular a MALDI analyzer, laser beam divided by sin θ, wherein θ is the MALDI analyzer laser beam incidence angle with respect to the sample plane. The invention also concerns processes of manufacture of the masks according to the invention, the use thereof for mass spectrometry, in particular MALDI, imaging of tissue sections, and a method for MALDI imaging of a tissue section using said masks.

The nature of the ion production method called Matrix Assisted Laser Desorption Ionization (MALDI) makes it naturally suitable for the analysis of crude samples such as tissues or tissue sections. In addition, since the desorption/ionization process is mediated in MALDI analysis by the irradiation of the sample by the laser beam, for any sample, the analyzed region is limited to the area irradiated by the laser beam. It is then possible to perform analyses in various points of the sample and to obtain in each point a spectrum representing ionic species present in this point.

Thus, by shifting the laser beam of a regular pitch defined by the user, the whole sample may be scanned, and a database comprising all spectra and their coordinates may be generated, which then allows to construct the expression map of any compound of known m/z ratio in the analyzed sample.

UV lasers used in MALDI imaging, and especially commercialized N₂ lasers emitting at 337 nm, have a laser beam section area generally ranging between 75×75 μm² and 200×200 μm² with a classical focusing system. For tissue imaging, the minimum distance between two points will have to be superior to the laser beam diameter, resulting in an image definition of at most the laser beam diameter (thus at best 75×75 μm), which corresponds to the irradiation of several cells in the tissue sample. Ideally, the image definition of a tissue sample should be or the order of a cell diameter (10-20 μm for small cells).

In the art, various methods have been tried to decrease the area irradiated by the laser beam for MALDI analysis.

Conventional methods aim at focusing the laser beam itself. This can be done either by using optical lenses adapted from the principle of Galilean telescope (1), or by using optical fibers (1-2). In both cases, it is possible to focus the laser beam down to a diameter of about 10 μm. However, these methods are very tricky and expensive to implement, especially using conventional, commercial MALDI devices. In addition, for a laser beam diameter below 25-30 μm, a very significant decrease in the quantity of ions produced in the gas phase is observed (2), which thus actually limits the image definition to 25-30 μm.

Another method involves adding an aperture (e.g. iridium) in the laser beam path. This method is much more convenient to set-up but unfortunately results in a significant decrease in the delivered energy (3), requiring then the use of solid state laser.

Alternatively, another method to decrease the diameter of the analyzed area involves the deposition of a regular pitch of matrix spots of defined diameter. Thus, even if the area irradiated by the laser beam is significantly larger, only the area coated with matrix should generate ions in the gas phase (4), however, this last point is not proved. Indeed, if the matter ejection is promoted by the incident beam impulse wave and not only by the absorption properties of matrix molecules, then the ejected matter could come from the whole irradiated area and not only from the matrix coated portion. Anyway, the use of this method is currently limited by the precision of microfluidic devices, which do not allow for the deposition of matrix spots with a diameter less than about 100 μm. In addition, the deposition of matrix spots is very time consuming (about 6-12 h for a whole rat brain section).

A summary of the above described methods of the prior art to increase the image definition by decreasing the area irradiated by the laser beam on a tissue sample with their advantages and drawbacks is presented in the following Table 1.

TABLE 1
Known methods to increase MALDI imaging resolution

| Method | Advantages | Drawbacks | Reference(s) |
|---|---|---|---|
| Focusing: | Precise focusing | Optical bench difficult | 1 |
| Galilean | | to implement | |
| telescope | | | |
| Focusing: | Precise focusing, | Intervention on the | 1-2 |
| optical fiber | “Flap-top” energetic | optical path inside | |
| | profile | the device | |
| Focusing: | Good focusing | Focusing limit around | 3 |
| diaphragm | | 50 μm | |
| Micro- | Possibility to have | Very time consuming | 4 |
| deposition of | matrix spots of | | |
| matrix spots | about 30 μm | | |

In view of the various drawbacks of known methods to increase MALDI imaging resolution, there is clearly a need for a new alternative, simpler, efficient and economical method to decrease the diameter of the analyzed area.

The inventors have found that the deposition on the tissue sample to analyze of “masks” made of an opaque material and displaying regularly spaced openings of a defined dimension allows for a significant decrease of the area actually irradiated by the laser beam, depending on the openings dimensions and the laser beam incidence angle. This way, the laser focusing can be decreased down to areas of about 15×75 μm², or even to about 15×50 μm², which is very close to a cell dimensions (about 20×20 μm²). In addition, the inventors also demonstrate that the use of such masks may induce a significant increase of the observed signal intensity, in particular for high m/z ratios. Thus, the masks are easily adaptable on any type of MALDI device, and allow to obtain images with a resolution close to a cell dimensions.

The present invention thus provides a mask for use in mass spectrometry, in particular MALDI, tissue section analysis, comprising a plate with an opaque external surface and having a thickness of less than 150 μm, said plate comprising regularly spaced openings, wherein in the plate upper plane, the diameter D of the largest circle comprising only one opening is superior to the diameter d of a mass spectrometer, in particular a MALDI analyzer, laser beam divided by sin θ, wherein θ is the mass spectrometer, in particular MALDI analyzer, laser beam incidence angle with respect to the sample plane.

As used herein, a “plate” means a substantially flat solid plate composed of two parallel and substantially plane external surfaces spaced by a thickness E (see FIG. 1A).

By a “substantially flat solid plate” is meant that the thickness E of the mask is highly inferior to the dimensions of the mask external surfaces.

By a “substantially plane external surface” is meant that a plane surface may be defined based on the mask external surface. In particular, the external surface may be a plane itself. Alternatively, the external surface may be microstructured, meaning that holes or asperities of dimensions highly inferior to the mask thickness E may be present on the mask external surface. In this case however, the negligible dimensions of the microstructure does not prevent from defining a mean plane external surface.

The two parallel and substantially plane external surfaces may display any geometric form in said plane, such as a rectangular, in particular a square (see FIG. 1A), or a circular or elliptic form. Preferably, the two parallel external plane surfaces display a rectangular, in particular a square, form (see FIG. 1A). The masks according to the invention were designed for use in mass spectrometry, in particular MALDI, analysis of tissue sections. As a result, the area of the plate should have dimensions that make it suitable for application on a tissue section deposited on a mass spectrometer, in particular MALDI, sample carrier. Usually, mass spectrometry, in particular MALDI, sample carriers display dimensions of about 5-10 cm×5-15 cm. Masks according to the invention should thus have dimensions inferior or equal to any MALDI sample carrier. In any case, for a mass spectrometer, in particular MALDI, sample carrier of dimensions larger than a mask according to the invention, several masks can be used if needed, juxtaposed onto the sample.

The masks according to the invention being intended for use in mass spectrometry, in particular MALDI, analysis, the plate material should be selected to be suitable for mass spectrometry, in particular MALDI, analysis, in particular, the plate material should be suitable for the desorption/ionization process of MALDI analysis to occur. As a result, a mask according to the invention should have an opaque external surface. By “opaque” is meant a material not transmitting the laser energy.

Preferably, a mask according to the invention should have an opaque and conductive external surface. By “conductive” is meant an electrically conductive material that is able to conduct electricity. More precisely, in the present invention, a “conductive material” is intended to mean either a semi-conductive or a strictly conductive (i.e. not semi-conductive) material. Advantageously, a mask according to the invention has an opaque and strictly conductive external surface. Alternatively, a mask according to the invention may have an opaque and semi-conductive external surface. By “an opaque and conductive external surface” is meant that the mask is globally opaque and that at least an external layer of the mask is made of a conductive (i.e. semi-conductive or strictly conductive) material. Preferably, a mask according to the invention can thus either be:
made of an opaque and conductive material,
made of an opaque, not conductive (i.e. insulator), material with other desirable properties and coated on its external surface by a conductive, opaque or not, material, or
made of a material with other desirable properties that is neither opaque nor conductive, and coated on its external surface by an opaque conductive material.

Examples of opaque conductive materials suitable in the invention include conductive metals (such as gold, nickel, chromium, aluminium, titanium, or tungsten); metal alloys such as stainless steel; carbon, silicon or polysilicon.

In a preferred embodiment, the plate is made of an opaque conductive material (see FIG. 1C). Preferably, said opaque conductive material is selected among silicon, or stainless steel. In a particular embodiment, the plate is constituted of a silicon wafer.

Alternatively, a mask according to the invention may be made of a non conductive but opaque material with desirable properties and coated on its external surface by a conductive material.

Alternatively also, a mask according to the invention may be made of another material with other desirable properties and coated on its external surface by an opaque and conductive material (see FIG. 1D).

Indeed, conductive materials are usually rather rigid materials, whereas it may be advantageous to have a mask made of a flexible material that may be moulded. The possibility to mould the mask may for instance be useful for an easy high throughput preparation of masks according to the invention on an industrial scale. In addition, a mask made of a flexible material may be less brittle and thus easier to manipulate for users. Suitable flexible materials are any flexible material that allows for the manufacture of mask with openings displaying the desired three dimensional structures and repartition upon moulding, including polymers, in particular some resists (such as SU-8, an epoxy resin of bisphenol A glycidyl ether polymer, CAS number 28906-96-9) or some silicone polymers or polysiloxanes, a group of inorganic or semi-inorganic polymers consisting of a silicon-oxygen backbone (—Si—O—Si—O—Si—O—) with side groups attached to the silicon atoms, such as in particular silicone resins or polydimethylsiloxane (PDMS, CAS number 63148-62-9). In this case, the moulded flexible plate has to be made conductive, and optionally opaque, depending on the first flexible material, to allow the ions transfer, which can be reached by metallization of the flexible moulded mask. An opaque conductive material, as defined above, in particular an opaque conductive metal, has thus to be coated on the external surface of the flexible plate. In the case of a metallic coating, if the metallic layer has to confer an opaque property, the thickness of the metallic layer has then to be sufficient (at least 100 nm).

Even when the mask is made of an opaque conductive material, it may be useful to coat the plate with a highly conductive material, such as gold, nickel, titanium or chromium. In a particular embodiment of any mask according to the invention, at least one side of said mask is further coated with a highly conductive material.

To allow for a normal desorption/ionization process and ions transfer, the thickness E of a mask according to the invention should be less than 150 μm, preferably less than 100 μm.

The masks according to the invention display regularly spaced openings. By “regularly spaced” is meant that the distance between the centers of two openings is constant. By the “center” of an opening is meant the point equidistant from all points on the circumference of the opening. The three dimensional structure of an opening depends on the upper and lower planes geometric forms, and on the inner surface form. There is no real constraint on the upper and lower planes geometric forms, or on the inner surface form. In particular, the inner surface form may be very diverse, and the section of this inner surface by a plane perpendicular to the plate plane may for instance have the form of a rectangle, a trapezoid (which may in some cases be isosceles or right), or other forms. Examples of the section of the inner surface of openings by a plane perpendicular to the plate plane are displayed in FIG. 1B.

Nevertheless, in a preferred embodiment, the three dimensional structure of an opening is such that the geometric form of any section of the opening by a plane parallel to the plate plane is a proportional transformation of the geometric form of the opening in the plate upper plane. The three dimensional structure of an opening can thus be characterized by the geometric form of the opening in the plate plane, and by the angle ox between the inner surface of the opening and the plate upper plane.

In addition, the various openings of a mask according to the invention may have identical or several different geometric forms, since the form of each opening is not a crucial feature. However, in a preferred embodiment, all openings of a mask display an identical form. By “identical form” is meant that the three dimensional structure of each opening is substantially identical, which means that, except for possible asperities due to a imperfect manufacture process, the desired three dimensional structure of each opening is identical.

Various geometric form of the opening in the plate plane can be used. Preferred geometric forms of the opening in the plate plane include rectangular, square, circular or elliptic. Thus, in a preferred embodiment, the openings display a rectangular or elliptic form, more preferably a square or circular form, in the plate plan. Examples of masks with square and circular openings are displayed in FIG. 1A.

The angle α between the inner surface of the opening and the plate upper plane is comprised between 0 and 90°, so that the geometric form of the opening in the plate lower plane has an area equal or inferior to that of the geometric form of the opening in the plate upper plane. FIGS. 1C and 1D show two suitable configurations for angle α. Preferably, α is comprised between 30 and 90°. In particular, a may be 90°, which corresponds to an inner surface of openings perpendicular to the plate plane, or 30°, 45°, 50°, 60° or 90°, which correspond to the incidence angles of commercial MALDI instruments. In a preferred embodiment, the inner surface of the openings and the plate plane thus form an angle of 30°, 45°, 50°, 60° or 90°.

The masks according to the invention were designed to improve the resolution of MALDI tissue sections imaging, by reducing the area irradiated by the MALDI laser beam. The dimensions of the openings of a mask according to the invention should thus be such that, depending on the openings form and the laser beam incidence angle, the area of the tissue section irradiated through the mask by the MALDI laser beam be inferior to the area that would be irradiated by the laser beam in the absence of the mask. Thus, in a mask according to the invention, the geometric form of the openings in the plate plane and the angle α between the inner surface of said openings and the plate upper plane are preferably such that, for a laser incidence angle θ comprised between 30° and 90°, between 40° and 80°, between 40° and 80°, between 40° and 70°, between 40° and 60°, or between 45° and 55°, the area of sample really irradiated by the laser beam is inferior to the area of the laser beam.

Depending on the angle α between the inner surface of the opening and the plate upper plane, on the incidence angle θ of the laser beam, and on the mask thickness E, it is possible that not the whole surface of sample corresponding to the form of the opening in the lower plate plane will be irradiated and that a fraction of this surface be in the shadow (shadowed area, see FIGS. 2 and 3). In particular, usual MALDI analyzers lasers have incidence angles θ between 30° and 90°. For masks with openings having inner surfaces forming an square angle α with the plate upper plane, a shadowed area will exist and the area really irradiated by the laser will be inferior to the opening area in the lower plate plane (see FIGS. 2 and 3). In particular, for a square opening and a laser beam diameter superior to the opening area, the incidence angle θ of the laser beam, the thickness E of the mask and the width l of the shadowed area are linked by the following formula: tan θ=E/l (see FIG. 3B). Calling L the width of the irradiated area, the shadowed area is then equal to 1×(L+1), while the irradiated area is equal to L×(L+1) (see FIG. 3B). In contrast, when α=θ, there is no shadowed area (see FIG. 4). The precise area actually irradiated by the laser in a precise configuration can be easily calculated by a man skilled in the art using the dimensions of the opening, the mask thickness E and the angles θ and α (see FIG. 3). For instance, for a mask displaying square openings of 100 μm side length and 65 μm or 100 μm thickness, and a laser incidence angle θ of 40°, 45°, 50° or 65°, the widths L of the area really irradiated are displayed in the following Table 2.

TABLE 2
Correlation between the mask thickness E, the laser incidence
angle θ and the width L (see FIG. 3) or the area really
irradiated by the laser for square openings
of 100 μm side length

| | Width L of the area irradiated | |
|---|---|---|
| | by the laser (μm) | |
| | Mask thickness E = | Mask thickness E = |
| Laser incidence angle θ | 65 μm | 100 μm |
| 60° | 67 | 46 |
| 50° | 45 | 17 |
| 45° | 35 | 0 |
| 40° | 13 | 0 |

By “the largest circle comprising only one opening” is meant the largest fictitious circle that comprises in its inner surface only one opening in the plate upper plane. This largest circle comprising only one opening is centered on the center of the opening geometric form and extends until touching the adjacent openings. Examples of this largest circle comprising only one opening for masks with square or circular openings are displayed in FIG. 1A. The fact that the diameter D of the largest circle comprising only one opening is superior to the diameter d of a MALDI analyzer laser beam divided by sin θ, wherein θ is the MALDI analyzer laser beam incidence angle with respect to the sample plane, ensures that when the laser is centered on a particular opening, only the sample accessible through this opening will be irradiated (see FIG. 1A).

The incidence angle θ of a MALDI analyzer can be obtained from the manufacturer, in particular from the documentation sold with the device. For instance, incidence angles θ of various commercial MALDI analyzers are provided in following Table 3.

TABLE 3
Usual commercial MALDI instruments incidence angles θ

| MALDI instrument name | Company | Incidence angle θ |
|---|---|---|
| Voyager Elite, | Applied Biosystems | 45° |
| Voyager Elite XL, | | |
| Voyager DE PRO, | | |
| Voyager DE-STR, or | | |
| Voyager DE-STR | | |
| autoflex II, | Bruker Daltonics | 30° |
| autoflex II TOF/TOF, | | |
| ultraflex II, and | | |
| ultraflex II TOF/TOF | | |

Concerning the laser beam diameter d, it may either be obtained from the manufacturer, or easily determined by measuring on a sample the area irradiated by the laser (i.e. the projection of the laser beam onto the sample surface). The following protocol may be used:
on a classical MALDI sample plate, depositing with a micropipette a few μL (generally 1 μL) of a matrix solution of α-cyano 4-hydroxycinnamic acid (CHCA) 15-20 mg/mL in acetone (saturated solution) in order to obtain a thin layer preparation,
drying at room temperature,
introducing the sample plate in the instrument, and irradiating on one fixed spot with the laser at classical experiments laser energy until the matrix layer is totally removed (3000-5000 laser shots on our instrument, depending on the laser energy, the laser type, the pulse duration and the laser repetition rate). The experiment can be performed several times on different spots,
removing the sample holder from the instrument and measuring the irradiated area by observing the sample under a microscope (optical, SEM . . . ) or scanning the sample and measuring with a drawing software.

Globally, since most current commercial MALDI analyzer lasers display diameters of about 75-150 μm, the diameter D of the largest circle comprising only one opening should for these lasers be superior to 75/sin θ−150/sin θ μm. MALDI lasers usually have an incidence angle θ comprised between 30° and 90°, corresponding to sin θ comprised between 0.5 and 1. Thus, for a conventional commercial MALDI laser of diameter comprised between 75-150 μm and depending on the incidence angle θ of the MALDI laser, the diameter D of the largest circle comprising only one opening should be superior to 75-300 μm. For masks that would be designed to be usable on any current commercial MALDI instrument, the diameter D of the largest circle comprising only one opening should be superior to 300 μm. More precisely, the values to which the diameter D of the largest circle comprising only one opening should be superior are listed in the following Table 4 for various incidence angles θ and laser beam diameter d.

TABLE 4
Value Dₘᵢₙ (μm) to which the diameter D of the largest circle
comprising only one opening should be superior for various
incidence angles θ and laser beam diameter d.

| | θ | | | | | | | |
|---|---|---|---|---|---|---|---|---|
| d | 30° | 40° | 45° | 50° | 60° | 70° | 80° | 90° |
| 75 μm | 150 | 117 | 106 | 98 | 87 | 80 | 76 | 75 |
| 100 μm | 200 | 156 | 141 | 131 | 115 | 106 | 102 | 100 |
| 125 μm | 250 | 194 | 177 | 163 | 144 | 133 | 127 | 125 |
| 150 μm | 300 | 233 | 212 | 196 | 173 | 160 | 152 | 150 |
| 175 μm | 350 | 272 | 247 | 228 | 202 | 186 | 178 | 175 |
| 200 μm | 400 | 311 | 283 | 261 | 231 | 213 | 203 | 200 |

However, it is clear that masks can be easily designed (by calculating d/sin θ)/and produced (using one of the processes described below) for use on any particular MALDI instrument, with a given incidence angle θ (obtained from the manufacturer) and diameter d (obtained from the manufacturer or determined as described above), with the condition that the diameter D of the largest circle comprising only one opening verifies D>d/sin θ. In particular, it should be understood that if lasers with a better focusing, and thus a lower diameter d, were manufactured and adapted to MALDI analyzers, then the diameter D of the largest circle comprising only one opening of a mask according to the invention could then be lower, provided it remains superior to d/sin θ.

It should be understood that, except for some limitations that have been above described, a high number of different masks can be useful and are therefore included in the scope of the present invention.

Particular masks useful for MALDI imaging comprised in the scope of the invention include masks with a plate made of a silicon wafer of thickness comprised between 50-150 μm displaying regularly spaced openings with a rectangular, in particular square, form, the length of the largest side being inferior to 500 μm, particularly preferred largest side lengths being 50, 100, 240 and 500 μm, and with an inner surface perpendicular to the plate plane.

With a laser displaying an incidence angle of 30°-60°, which is the common case, such masks make it possible to have a sample irradiated area inferior to the area that would be irradiated by the laser beam in the absence of the mask (see Table 2).

In addition, such masks also do not lead to a decrease in the signal intensity of detected ions, but surprisingly may even result in a significant increase in said signal intensity, in particular for high m/z ratios (i.e. m/z>3000).

Masks according to the invention has been intended for mass spectrometry analysis of samples, in particular for MALDI analysis of tissue samples. However, they may be used with other mass spectrometers. In particular, the matrix is essential to MALDI desorption/ionization. It acts as an energy receptor for the pulsed laser beam, using that energy to desorb cocrystallized analytes. However, the technique produces a large amount of matrix background ions, which can obscure or suppress small mass ions and thus limiting the use of the technique for the quantitation of small molecules. Several strategies have been proposed to improve the results of analysis.

First, laser Desorption/Ionization On porous Silicon (DIOS) mass spectrometry, has been recently reported (5). Porous silicon (PSi) showed the feasibility as a matrix-free desorption/ionization substrate, where the absence of matrix-related ions extends the observable mass range to small molecules. Hydrogen-terminated porous silicon (PSi—H) surfaces are obtained by electrochemical dissolution of crystalline silicon in HF-based solutions. This technique is well-established. Flat thin films of PSi with reasonably well-defined pore morphologies can be prepared from a single-crystal silicon wafer by chemical and/or electrochemical etching in HF-based solutions. The resulting porosity, pore size, and PSi layer thickness depend on the etching conditions such as the current density, the composition of the etching solution, and the etching time as well as the type, doping level, and orientation of the substrate.

Still more recently, dense arrays of crystalline silicon nanowires (SiNWs) have been used as a platform for laser desorption/ionization mass spectrometry of small molecules, peptides, protein digests, and endogenous and xenobiotic metabolites biofluidics (6). Under optimized conditions sensitivities down to the attomole level have been achieved. Silicon nanowires (SiNWs) can be prepared using the so-called Vapor-Liquid-Solid (VLS) technique. The technique consists on chemical decomposition of silane gas (SiH₄) catalyzed by Au NPs at high temperatures (440-540° C.). In this process, the diameter of the nanowires is determined by the diameter of the catalyst particles and therefore, the method provides an efficient means to obtain uniform-sized nanowires. SiNWs with a narrow size distribution were obtained by using well-defined catalysts (gold nanoparticles). The technique is easily adaptable for large areas synthesis with relatively low costs. This technique permits to grow SiNWs in a controlled fashion on different substrates (7). Other semiconductor nanostructures such as zinc oxide (ZnO), tin oxide (SnO₂), gallium nitride (GaN) and silicon carbide (SiC) nanowires were also found to be efficient for energy transfer matrix for quantitative analysis of small molecules (8).

Another example based on gold nanoparticles (Au NPs) for matrix assisted laser desorption/ionization of peptides has been reported in the literature (9). Gold nanoparticles (Au NPs) may be prepared by various technologies. First, Au NPs can be prepared by a physical methodology, consisting of thermal evaporation of thin Au film and its subsequent annealing. In particular, thin silica films deposited by PECVD technique on gold films without any adhesion layer exhibit high stability [13-15]. Furthermore, these silica films may be used to deposit thin films of gold. For instance, a 3 nm thin Au film may be formed on PECVD-deposited SiOₓ on silicon substrate after thermal annealing for 10 min at 400° C. The thermal annealing allows the Au thin film to self-assemble into a dense and uniform array of nanoparticles. The resulting Au nanoparticles exhibit an average diameter in the range of 10-25 nm. Decreasing the thickness of the initial Au film leads to a slight decrease of the nanoparticle average diameter. Alternatively, monodispersed gold colloid solutions having particle sizes between 2.5-50 nm are commercially available. Drop casting technique may thus be used for gold nanoparticles deposition on amine or thiol-terminated surfaces. Another suitable technology to generate Au NPs is E-beam lithography, which allows the formation of gold nanostructures with controlled size, shape and position.

Porous silicon and gold nanoparticles composites might also be used. PSi/Au NPs substrates may be obtained using two different approaches:

1. Gold nanoparticles are deposited on amine-terminated PSi substrates prepared by silanization of the oxidized surface with aminopropyl triethoxysilane. The strong interaction between NH₂ groups and Au nanoparticles directs a controlled deposition of the nanoparticles on the surface. The density of the nanoparticles on the surface is controlled by the initial dilution of the gold colloid. This technology permits to obtain an amine-terminated PSi surface coated with 10 nm average diameter gold nanoparticles, showing a good and a long-term stability in aqueous solution. This is an important asset for further modification of the nanoparticles to introduce chemical or biochemical functionalities on the surface.

2. Thermal evaporation of thin Au films (1 -4 nm) on freshly or oxidized PSi surfaces leads to Au nanoparticles deposition on the PSi surface.

Formation of PSi layers requires holes supply from the silicon substrate and thus the preparation of PSi films on n-type crystalline silicon necessitates white light irradiation of the surface to generate holes. Because of the light stimulation of electrochemical etching of n-type Si, patterns or arrays of PSi can be obtained by use of simple mask. Furthermore, patterns of PSi can also be directly formed on p-type silicon substrate using a patterned resist stable in the etching solution (HF/EtOH).

In a similar way, patterns of PSi/Au NPs may be fabricated by thermal evaporation of thins Au films or Au NPs adsorption on amine-terminated structures.

SiNWs arrays may be formed by pre-patterning of the gold nanoparticles catalyst and subsequent chemical vapor decomposition of SiH4 at high temperature.

All these technologies permitting not to be dependent on a matrix as in MALDI mass spectrometry, and thus to improve analysis of small molecules may be incorporated into masks according to the invention, using the protocols described above.

In a particular embodiment of masks according to the invention as described above, masks thus further display a layer of porous silicon; semi-conductor nanowires arrays, in particular silicon nanowires; gold nanoparticles arrays; or porous silicon and gold nanoparticles composite arrays on the external surface intended to be in contact with the sample, and optionally also in part or entirety of the openings inner surface. Preferably, such masks display a layer of porous silicon; gold nanoparticles arrays; or porous silicon and gold nanoparticles composite arrays on the external surface intended to be in contact with the sample, and optionally also in part or entirety of the openings inner surface. Still preferably, such masks display a layer of porous silicon on the external surface intended to be in contact with the sample, and optionally also in part or entirety of the openings inner surface.

Such masks are thus suitable for use in mass spectrometry without the need to resort to a matrix as in MALDI technology. In particular, such masks are suitable for use in DIOS mass spectrometry.

Such masks may further be chemically functionalized. Freshly prepared porous silicon or silicon nanowires surfaces are hydrogen-terminated. Different organic monolayers on PSi—H or SiNWs-H can be prepared using existing and developed techniques. It consists on hydrosilylation reaction of different alkenes and aldehydes with PSi—H surface to yield organic monolayers covalently bonded to the surface through stable Si—C and Si—O—C bonds. Organic molecules with different functional end groups can be synthesized and covalently attached to the surface under thermal conditions. The structure of the organic molecule used during the chemical process determines the wetting properties (hydrophobic/hydrophilic) of the surface. Moreover, partial oxidation of the PSi surface followed by chemical derivatization allows the preparation of surfaces with different chemical compositions. Different ligands that recognize specifically certain bacteria or hazardous material can be synthesized and attached to the PSi surface. Hydrogen-terminated PSi or SiNWs surfaces can also be oxidized by several means: thermal, electrochemical, chemical, and UV-ozone. The resulting surfaces contain high concentration of surface hydroxyl groups that can then be easily coupled to trichloro or trialkyloxysilanes (10).

Au nanoparticles chemical functionalization can be accomplished using the well-known chemistry of alkanethiol assembly (10).

The invention further concerns a process for manufacturing a mask according to the invention, comprising:
a) providing a plate made of an opaque conductive material and having a thickness of less than 150 μm,
b) creating openings in said plate, wherein in the plate plan, the diameter D of the largest circle comprising only one opening is superior to the diameter d of a MALDI analyzer laser beam divided by sin θ, wherein θ is the MALDI analyzer laser beam incidence angle with respect to the sample plane.

By “creating openings” is meant the fact to make holes in the mask displaying the desired openings three dimensional structure. The openings may be created using any suitable technology able to result in openings of the desired three dimensional structures.

In an advantageous embodiment, the creating said openings comprise:
i) cleaning the plate,
ii) coating the plate with a positive or negative photoresist,
iii) irradiating the coated plate with UV through a chromium coated glass protection displaying such a configuration that the areas corresponding to the positions of the desired mask openings correspond either to chromium coated glass protected areas (in the case of a negative photoresist) or to not chromium coated glass protected areas (in the case of a positive photoresist),
iv) removing the photoresist in the areas corresponding to the desired openings using a development solution,
v) attacking the areas corresponding to the desired openings using dry etching to create the openings, and
vi) cleaning the obtained mask to remove asperities.

The above method for creating openings may further comprise an optional step i1) between steps i) and ii) in which aluminium is deposited onto the plate. Aluminium deposition may be made notably by sputtering or evaporation.

Such an optional step is advised for deep wet etching (by ICP), thus particularly in the case of thick membranes since resins do not resist for a long time the wet etching.

In this case, the aluminium etching is also made by the development solution in step iv).

According to the invention, a “photoresist” is a photosensitive, chemically resistant material that may be used to protect areas of a material that will be subsequently etched. Photoresist are usually used to mask areas of printed circuit board blanks, but are also useful in the present invention.

By a “positive” photoresist is meant a type of photoresist that has a higher developer dissolution rate after exposure to UV light, which changes the chemical structure of the photoresist, making it more soluble in developer solution. The use of a development solution then permits to dissolve and remove only the photoresist areas that have been exposed to light. The chromium coated glass protection, therefore, contains an exact copy of the pattern which is to remain on the obtained mask according to the invention. A positive photoresist usually contains a non-photosensitive base phenolic resin (condensation polymers of aromatic alcohols and formaldehyde) such as Novolak (phenol formaldehyde polymer, CAS number 9003-35-4), a photosensitive dissolution inhibitor such as a diazonaphthaquinone-derived compound, and a coating solvent such as n-butyl acetate, xylene, propylene-glycol-monomethyl-ether acetate (PGMEA), or 2-ethoxyethyl acetate. It may additionally contain antioxidants, radical scavengers, amines to absorb O₂ and ketenes, wetting agents, dyes to alter the spectral absorption characteristics, adhesion promoters, and/or coating aids. Common positive photoresists are thus based on a mixture of Diazonaphthoquinone (DNQ) and Novolak resin (a phenol formaldehyde polymer) in an acceptable solvent as described before. Other suitable positive photoresist include AZ® 1518, AZ® 4562, or AZ® 9260 photoresists (mainly based on a mixture of Novolak resin, a photoactive agent (PAC) such as diazonaphthoquinone (DNQ) and of solvent (PGMEA, propylene-glycol-monomethyl-ether acetate, CAS number 108-65-6)) available notably from AZ Electronic Materials USA Corp (70 Meister Avenue, Somerville, N.J. 08876, USA) or Shipley (Coventry, UK).

By a “negative” photoresist is meant a type of photoresist that becomes relatively insoluble to developer when exposed UV light, which causes the negative photoresist to become polymerized, and more difficult to dissolve. The use of a development solution then permits to dissolve and remove only the photoresist areas that have not been exposed to light. The chromium coated glass protection used with negative photoresists, therefore, contain the inverse (or photographic “negative”) of the pattern to be transferred to the mask according to the invention. A negative photoresist usually contains a non-photosensitive substrate material (about 80% of solids content, usually cyclicized poly(cis-isoprene)), a photosensitive cross-linking agent (about 20% of solids content, usually a bis-azide ABC compound), a coating solvent (usually a mixture of n-butyl acetate, n-hexyl acetate, and 2-butanol). It may additionally contain antioxidants, radical scavengers, amines; to absorb O₂ during exposure, wetting agents, adhesion promoters, coating aids, and/or dyes. Common negative photoresists are based on an epoxy based polymer, including Microchem SU-8 (epoxy resin (CAS number 28906-96-9) with mixed triarylsulfonium/hexafluoroantimonate salt and propylene carbonate formulated in gamma butyrolactone) and SU-8 2000 (same composition that SU-8 but formulated in cyclopentanone) photoresists. Other suitable negative photoresist include AZ® 5214 or AZ® nLoF photoresists (mainly based on a mixture of Novolak resin, a photoactive agent (PAC) such as diazonaphthoquinone (DNQ) and of solvent (PGMEA, propylene-glycol-monomethyl-ether acetate)) available notably from AZ Electronic Materials USA Corp (70 Meister Avenue, Somerville, N.J. 08876, USA) or Shipley (Coventry, UK).

By a “development solution” or “developer solution” is meant a solution used to resolve an image after exposure. For a positive photoresist the developer has a higher attack rate on the exposed portion of a photoresist than on an unexposed portion of the photoresist. In particular, DNQ-Novolak resists are developed by dissolution in a basic solution (usually 0.26N tetra-methyl ammonium hydroxide in water). For a negative photoresist the developer has a high attack rate on the unexposed portion of the photoresist than on the exposed portion of the photoresist. Negative photoresist developers are solvents which swell the resist, allowing uncross-linked polymer chains to untangle and be washed away. A sequence of solvents is often used to keep the swelling reversible. For instance, negative SU-8 photoresist can be developed using Microchem SU-8 developer, ethyl lactate or diacetone alcohol.

According to the invention, “dry etching” refers to the removal of material by exposing the material to a bombardment of ions that dislodge portions of the material from the exposed surface. Dry etching typically etches directionally or at least anisotropically, which permits to obtain in the masks according to the invention openings with an internal surface substantially perpendicular to the plate planes. Dry etching technologies notably include non-plasma based dry etching, such as Xenon Difluoride (XeF2) Etching or Interhalogen (BrF3 & ClF3) Etching, or plasma based dry etching, such as Reactive Ion Etching (RIE), Deep Reactive Ion Etching (DRIE), Electron Cyclotron Resonance (ECR), or Inductively Coupled Plasma (ICP).

Preferably, dry etching in step v) of the above described advantageous embodiment protocol is performed using Inductively Coupled Plasma (ICP), more preferably using Bosch process (as described in DE4241045).

A precise protocol to create openings according to this embodiment is described in Example 1.1.

In another advantageous embodiment, creating the openings comprises:
i) cleaning the plate,
ii) coating the plate with a silicon oxide or nitride,
iii) coating the plate with a positive or negative photoresist,
iv) irradiating the coated plate with UV through a chromium coated glass protection displaying such a configuration that the areas corresponding to the positions of the desired mask openings correspond either to chromium coated glass protected areas (in the case of a negative photoresist) or to not chromium coated glass protected areas (in the case of a positive photoresist),
v) removing the photoresist in the areas corresponding to the desired openings using a development solution,
vi) reporting the openings on the silicon oxide or nitride oxide by a Reactive Ion Etching (RIE) etching (plasma CHF3/CF4),
vii) attacking the areas corresponding to the desired openings using wet etching to create the openings, and
viii) cleaning the obtained mask to remove asperities.

This alternate method for creating openings may further comprise an optional step after step viii) or between steps v) and vi) consisting in thinning down the plate to the thickness of the mask wished (and thus according to the deliberate opening). This operation is possible by wet etching (KOH, TMAH) or dry etching (ICP).

According to the invention, “wet etching” refers to the removal of material by immersing the material to be etched in a liquid bath of chemical etchant. Wet etchants fall into two broad categories; isotropic etchants and anisotropic etchants. Isotropic etchants attack the material being etched at the same rate in all directions. Anisotropic etchants attack the silicon wafer at different rates in different directions, and so there is more control of the shapes produced. Some etchants attack silicon at different rates depending on the concentration of the impurities in the silicon (concentration dependent etching). Usual anisotropic wet etchants are well known to the person skilled in the art and include potassium hydroxyde (KOH), tetramethylammonium hydroxide (TMAH), ethylene diamine pyrocatechol (EDP), and ethylenediamine pyrocatechol and water (EPW). In the context of the invention, anisotropic wet etchants, and in particular KOH and TMAH, are preferably used for wet etching performed in step vii) of the above described other advantageous embodiment. These anisotropic etchants permit to obtain in the masks according to the invention openings displaying an angle α between the internal surface and the plate planes strictly inferior to 90°. In particular, in the case of a silicon wafer (<100> orientation) when using TMAH, the angle a is 54.7°. The section of the inner surface by a plane perpendicular to the plate plane has then the form of a trapezoid (V openings). A precise protocol to create such openings is described in Example 1.2.

In still another advantageous embodiment, the openings are created using a micro machining technology, such as laser microsurgery, electrochemistry, or hot embossing. A person skilled in the art of micro machining technology will know the suitable protocols to use in a particular case.

The invention also concerns another process for manufacturing a mask according to the invention, comprising:
a) providing a rigid mould displaying the desired mask configuration,
b) casting a flexible material into the rigid mould to obtain a flexible mask with the desired configuration, and
c) coating the external surface of the resulting mask by a conductive material, said conductive material being also opaque if the flexible material used in step b) is not opaque.

The flexible material cast into the mould to obtain said flexible mask may be any flexible material suitable for micro-moulding, meaning any flexible material that may be cast into the mould and allows for the manufacture of mask with openings displaying the desired three dimensional structures and repartition upon moulding. For instance, any of the previously described suitable flexible materials may be used. In a preferred embodiment of the above described process of manufacture comprising a moulding step, the flexible material is a silicone polymer or a photoresist, as defined above. In particular, SU-8 negative photoresist may be used as flexible material. Advantageously, PDMS is used as flexible material.

The coating of the external surface of the resulting flexible moulded mask by an opaque conductive material may be performed using any suitable technology that allows for the deposition of a globally regular layer of opaque conductive material on the external surface, without significantly altering the three dimensional structure of the openings. Suitable technologies include for instance sputtering and evaporation. The opaque conductive material used may be any material displaying the properties previously defined as “opaque” and “conductive”, in particular any of the previously mentioned opaque conductive material. When the flexible material used is not opaque and the coating aims at conferring both conductivity and opacity, the thickness of the opaque and conductive material layer coated onto the flexible mask should be sufficient to confer opacity (more than 100 nm for a metallic coating).

Still another process to make a mask according to the invention, when the flexible material used for the plate is a photoresist, consists in providing a plate made of a photoresist and directly creating openings by irradiation through a chromium coated glass protection displaying such a configuration that the areas corresponding to the positions of the desired mask openings correspond either to chromium coated glass protected areas (in the case of a negative photoresist) or to not chromium coated glass protected areas (in the case of a positive photoresist). For instance, Microchem provides flexible films of SU-8 photoresist named XP MicroForm™ 1000. The coating of the external surface of the resulting flexible mask by an opaque conductive material may be performed using any suitable technology that allows for the deposition of a globally regular layer of opaque conductive material on the external surface, without significantly altering the three dimensional structure of the openings, as described above.

The masks according to the invention were designed to reduce the dimensions of the area of a sample that may be analyzed to dimensions close to the dimensions of a single cell. The masks may thus be used to improve the image resolution of MALDI analysis of tissue sections, the reason why they were designed by the inventors, but also to other applications in which a reduction of the area of sample analyzed is necessary or useful. For instance, the masks according to the invention may be used for MALDI imaging of chosen individual cells of interest, for instance a particular cell type, within a tissue section sample, or even, if the resolution is sufficient, to analyze a particular region within an individual cell. Alternatively, the masks may also be used for MALDI analysis of a sample carrier on which individual cells have been deposited. Each cell, or even a particular region within each individual cell, may then be analyzed separately.

The invention thus concerns the use of a mask according to the invention for MALDI analysis of a tissue section or of individual cells deposited onto a sample carrier. In a preferred embodiment, the masks according to the invention are used for MALDI analysis of a tissue section. By “analysis of a tissue section” is meant either the imaging of an area of the tissue section, or only the analysis of particular areas of the tissue section comprising cells of interest, for instance cells of a desired cell type. Preferably, the masks according to the invention are used for MALDI imaging of a chosen area of the tissue section. Alternatively, the masks according to the invention are used for MALDI analysis of particular areas of the tissue section comprising cells of interest, for instance cells of a desired cell type. In another preferred embodiment, the masks according to the invention are not used on a tissue section sample, but on a sample carrier onto which individual cells have been deposited.

The masks according to the invention might even be used in other applications that MALDI analysis. For instance, the masks according to the invention might be used for fluorescence microscopy analysis of a tissue section or of individual cells deposited onto a sample carrier. Indeed, the analysis of a restricted area may decrease the background fluorescent noise and thus improve the quality of the analysis.

The invention also concerns a method for MALDI imaging of a tissue section, comprising:
a) Providing a tissue section sample on a MALDI sample carrier,
b) Depositing a suitable MALDI matrix onto the surface on said tissue section sample,
c) Depositing a mask according to the invention directly onto the surface of said matrix coated tissue section sample,
d) Analyzing said tissue section sample in each mask opening using a MALDI mass spectrometer and storing all obtained spectra, and
e) Constructing the expression map of any desired compound of known m/z ratio using said stored spectra.

According to the invention, a “tissue section” preferably has the following properties: it may be frozen, fixed or fixed and paraffin-embedded, its thickness is preferably in the order of a mammalian cell diameter, thus comprised between 5 and 20 μm. In the case of a frozen section that was obtained from a frozen tissue using a cryostat, OCT (optimal cutting temperature polymer) is preferably used only to fix the tissue but the frozen tissue is not embedded in OCT, so that tissue sections were not brought into contact with OCT. The tissue section may then be transferred on a “MALDI sample carrier” composed of any material suitable for further MALDI analysis, including metals, inorganic or organic materials, such as gold, steel, glass, nylon 6/6, silicon, plastic, polyethylene, polypropylene, polyamide, polyvinylidenedifluoride or a glass slice of any thickness coated with conductive metal keeping transparency properties such as nickel or ITO.

By a “suitable MALDI matrix” is meant any material that, when mixed with the analyte, generates crystalline matrix-embedded analyte molecules that are successfully desorbed by laser irradiation and ionized from the solid phase crystals into the gaseous or vapour phase and accelerated as molecular ions. Commonly used MALDI-MS matrices are generally small, acidic chemicals absorbing at the laser wavelength, including nicotinic acid, cinnamic acid, 2,5-dihydroxybenzoic acid (2,5-DHB), α-cyano-4-hydroxycinnamic acid (CHCA), 3,5-dimethoxy-4-hydroxycinnamic acid (sinapinic acid or SA), 3-methoxy-4-hydroxycinnamic acid (ferulic acid), 3,4-dihydroxycinnamic acid (caffeic acid), 2-(4-hydroxyphenylazo)benzoic acid (HABA), 3-hydroxy picolinic acid (HPA), 2,4,6-trihydroxy acetophenone (THAP) and 2-amino-4-methyl-5-nitropyridine. Protocols for the preparation of these matrices are well-known in the art, and most of these matrices are commercially available. Current commonly used matrices for peptide/protein analysis include α-cyano-4-hydroxycinnamic acid (CHCA), 2,5-dihydroxybenzoic acid (2,5-DHB) and sinapinic acid (SA). DNPH is 2,4-Dinitrophenylhydrazine is a reactive matrix and is used for aldehydes and ketones detection.

In addition, other matrices, which are especially suitable for direct tissue sections analysis, may be used in the above described method. Such matrices notably include ionic matrices. A “ionic matrix” is a complex constituted of a charged matrix and a counter-ion. As MALDI matrices are usually acidic, such ionic matrices are usually prepared by an acid-base reaction between an acid conventional MALDI matrix and an organic base, leading to a proton exchange between the two compounds and resulting in a [Matrix⁻Base⁺] complex. Despite the usual acidic properties of matrices, some basic matrices also exist, such as the 2-amino-4-methyl-5-nitropyridine (2A4M5NP) matrix. Ionic matrices may thus also be prepared by an acid-base reaction between an acidic and a basic conventional matrix, resulting in a [Acidic matrix⁻Basic matrix⁺] complex after proton exchange. Schematically, the synthesis of a ionic matrix may be performed by mixing equimolar amounts of the two acidic and basic compounds in an organic solvent, such as for instance methanol. After one hour of stirring at room temperature, solvent is evaporated and the resulting ionic matrix is dissolved in an acetronitrile/water solution before use for MALDI analysis. Particularly advantageous ionic matrices for the implementation of the invention comprise [CHCA⁻ANI⁺], [CHCA⁻DANI⁺], and [CHCA⁻2A4M5NP⁺], wherein ANI and DANI respectively refer to aniline and N,N-dimethylaniline. More details about ionic matrices useful for MALDI tissue sections analysis are provided in U.S. provisional patent No. 60/687,848.

After matrix deposition, a mask according to the invention is directly deposited on the matrix coated tissue section, as indicated in FIG. 5.

The MALDI analysis of each opening is then performed and each resulting signal intensity versus m/z ratios profiles are stored.

An “expression map” of any compound of known m/z ratio may then be constructed. Indeed, by analysis the signal intensity at the desired m/z ratio in all stored profiles, a two dimensional image of the expression level of the compound displaying this particular m/z ratio may be constructed.

The main goal of the invention id to provide products, processes of manufacture thereof and method of use thereof to improve MALDI analysis of tissue sections resolution. The only apparent drawback of the masks according to the invention might be the fact that the openings have to be separated by a distance enough to prevent that when the laser beam is centered on a particular opening, it cannot irradiate the sample accessible in an adjacent opening. The above described method thus allows for more precise, smaller sample areas to be analyzed in each opening, but the analyzed points are necessarily separated by a certain distance. However, this fact can be easily corrected using the masks according to the invention by inserting in the above described method for MALDI imaging of a tissue section at least one repetition of a d′) step comprising:
i) Moving the mask according to the invention or the tissue section sample so that in the new position, the mask openings are placed upon a sample area that had not been previously irradiated, and
ii) Analyzing said tissue section sample in each mask opening using a MALDI mass spectrometer and storing all obtained spectra.

The MALDI analyzer may thus be adapted so that the sample is fixed and the mask is mobile, or the contrary. Step d′) may be repeated as many times as necessary to have a significant proportion of the sample area analyzed. Advantageously, the mask is fixed and the sample is mobile.

In a preferred embodiment, the openings of the mask are spaced so that the distance between two adjacent openings, defined as the minimal distance between two points of the adjacent openings circumference, is equal to n.½(distance between the center of two adjacent openings), wherein n is an integer. This way, an integer number n of opening areas is available between two adjacent openings. For instance, when n is one, the area of one opening is available between two adjacent openings (see FIG. 5). Step d) has then to be performed once and step d′) three times (four positions of analysis in total) to have a significant, or even complete depending on the opening geometric form, proportion of the sample area analyzed (see FIG. 5).

The use of a mask according to the invention for MALDI imaging of tissue sections, and especially of the above described method for MALDI imaging of a tissue section allows for a higher resolution of expression maps by reducing in each point analyzed the area irradiated by the laser. In addition, the presence of a mask according to the invention on the tissue section sample does not lead to any signal intensity decrease, and may even result, although the precise mechanism leading to this surprising finding is not completely elucidated, in a significant increase in the signal intensity, in particular for high m/z ratios (i.e. m/z superior to about 3000). This result is particularly interesting for MALDI imaging of tissue sections, since most of the prior art technologies used to improve the resolution tend to result in a signal decrease. Finally, a huge advantage of the masks according to the invention is certainly their easy adaptability on any MALDI analyzer, since their use does not involve any material modification or investment in new equipment. The mask only has to be deposited onto the matrix coated tissue section before analysis.

Having generally described this invention, a further understanding of characteristics and advantages of the invention can be obtained by reference to certain specific examples and figures which are provided herein for purposes of illustration only and are not intended to be limiting unless otherwise specified.

### DESCRIPTION OF THE DRAWINGS

FIG. 1. General description of a mask according to the invention. A. A square plate of thickness E made of or coated by conductive opaque material is displayed with square or circular openings regularly spaced such that the diameter D of the largest circle comprising only one opening is superior to the diameter d of a MALDI analyzer laser beam divided by sin θ, wherein θ is the MALDI analyzer laser beam incidence angle with respect to the sample plane. B. Examples of forms of the section of the inner surface of openings by a plane perpendicular to the plate plane C. Perpendicular section of a mask according to the invention with a particular openings three dimensional structure. In this configuration of a mask according to the invention, the plate is made of an opaque conductive material, and the angle α between the inner surface of the opening and the plate upper plane is 45°. D. Perpendicular section of another mask according to the invention with another particular openings three dimensional structure. In this configuration of a mask according to the invention, the plate is made of a flexible material and coated by an opaque conductive material, and the angle α between the inner surface of the opening and the plate upper plane is 90°.

FIG. 2. Matter ejection for a laser beam incidence angle θ=45° and an angle between the inner surface of the opening and the plate upper plane α =90° depending on the mask thickness E and openings dimension O. A. For E≧O, the laser beam does not reach the sample and no matter is ejected. B. For E<O, the laser beam reaches a fraction of the sample area not protected by the mask and matter comprised in this irradiated area is ejected and analyzed.

FIG. 3. Presence of a shadowed area when the laser beam incidence angle is inferior to 90° and the lateral surface of the openings is perpendicular to the plate plan. A. The tissue, the mask and the laser beam are displayed. Due to the laser beam incidence angle, the area irradiated by the laser beam through the mask is smaller than the total area of sample not protected by the mask. The remaining accessible sample area stays in the shadow. The irradiated and shadowed areas are displayed. B. Upper view of a rectangular opening irradiated by a laser beam. The instrument axis, the laser beam incidence angle θ, the width of the shadowed area l, the width of the irradiated area L, and the mask thickness E are displayed. θ, E and l are related by the equation tan θ=E/l. The shadowed area is then equal to 1×(L+1), and the irradiated area to L×(L+1).

FIG. 4. Example of a mask displaying openings with a lateral surface forming an angle of 45° with the mask plan, which is particularly suitable for use with a MALDI analyzer comprising a laser beam with an incidence angle of 45°. In this configuration, the whole accessible sample area is irradiated, there is no shadowed area.

FIG. 5. Scheme showing an example of a method according to the invention on a tissue section sample. A tissue section sample is deposited on a MALDI conductive sample carrier (step a)), coated with a MALDI matrix (the matrix is deposited onto the tissue sample and let dry, step b). Then, a mask according to the invention is directly applied on the tissue section sample before MALDI analysis (step c)) and MALDI analysis is performed in each opening (step d)).Then, step d′) is repeated three times as displayed on the scheme to have a complete MALDI image of an area of the tissue section sample. Data analysis (step e)) of obtained spectra at a given m/z ratio gives an expression map of the compound displaying this m/z ratio in the area of the sample analyzed.

FIG. 6. Principle of a process of manufacture of a mask according to the invention with square openings and an angle α between the inner surface of the opening and the plate upper plane of about 90°. Step 1: a thin silicon wafer (thickness of about 100 μm) is coated with the negative photoresist SU-8. Step 2: the SU-8 negative photoresist coated silicon wafer is UV irradiated through a chromium coated glass protection displaying shapes of the desired configuration corresponding to the future mask openings. Step 3: the UV irradiated SU-8 coated silicon wafer is exposed to a development solution (Microchem SU-8 developer) which removes the unexposed SU-8 negative photoresist in the areas protected by the glass protection. Step 4: the areas not protected by the remaining SU-8 negative photoresist are attacked by Inductively Coupled Plasma (ICP) using Bosch process (as described in DE4241045), resulting in openings with the same configuration as the glass mask.

FIG. 7. Scanning electron microscopy (SEM) photographs of masks according to the invention with square openings and an angle α between the inner surface of the opening and the plate upper plane of about 90° for various openings dimensions. a. 50 μm. b. 100 μm.

FIG. 8. Principle of a process of manufacture of a mask according to the invention with square openings and an angle α between the inner surface of the opening and the plate upper plane of about 55°. Step 1. Nitride (Si₃N₄) is deposited on both faces of the silicon wafer. Step 2. AZ 5214 photoresist (Shipley) is deposited on the upper surface (speed 3000, acceleration 1000, duration 7 seconds), and the coated wafer is cured at 120° C. during 60 seconds. The AZ 5214 photoresist coated upper surface of the silicon plate is then UV irradiated through a chromium coated glass protection having circular openings with a diameter of 200 and 400 μm, and the plate is cured at 120° C. during 60 seconds. The AZ 5214 photoresist coated upper surface of the silicon plate is then UV irradiated on the whole surface during 60 seconds. Step 3. the AZ 5214 photoresist layer is removed in areas not protected by the chromium coated glass protection, using a pure metal ion free (MIF) developer during 20 seconds. The plate is rinsed using deionized (DI) water. Step 4. The circular openings are reported on nitride (Si₃N₄) by a Reactive Ion Etching (RIE) etching (plasma CHF3/CF4). Step 5. The areas corresponding to the desired openings are attacked using wet etching with TMAH (speed 0.5 μm/minute) to create the openings. Depending on the desired thickness, wet etching is performed during 200 to 480 minutes. Due to the crystalline structure of silicon, the circular areas give rise to square openings into silicon. Step 6. Both Si₃N₄ coated faces of the plate are then attacked using RIE (125W, 50 mTorr, CF4:40, CHF3:40, about 7 minutes 30 seconds). Step 7. The lower face of the plate is etched using ICP-STS to lower total thickness and the resulting mask is cleaned using the “piranha” solution.

FIG. 9. A. Scheme of a mask according to the invention with square openings and an angle α between the inner surface of the opening and the plate upper plane of about 55°. B. and C. Scanning electron microscopy (SEM) photograph (lateral view after cutting through one opening) of a mask presenting square V openings of 270 μm external dimension, 103 μm internal dimension, 119 μm thickness and α=54.7° machined from a silicon wafer using protocol described in example 1 at two distinct scales: B. 1 cm=34,246 μm. C. 1 cm=11,765 μm.

FIG. 10. A. Scanning electron microscopy (SEM) photograph of a mask presenting dense array of square V 100*100 openings on 1 cm². B. Scanning electron microscopy (SEM) photograph of some openings with 95 μm external dimension, 36 μm internal dimension, 42 μm thickness and α=54.7° machined from a silicon wafer using the protocol described in example 1.

FIG. 11. Standards MALDI analysis using masks according to the invention. A mix of standard peptides of known m/z ratio was deposited on a MALDI sample carrier. After matrix deposition, a mask according to the invention with A. a thickness of about 65 μm and square openings having a side dimension of 500 μm, B. a thickness of about 65 μm and square openings having a side dimension of 240 μm, C. a thickness of about 100 μm and square openings having a side dimension of 100 μm, and D. a thickness of about 100 μm and square openings having a side dimension of 50 μm. The signal intensities for m/z ratios of 500 to 10000 are displayed.

FIG. 12. Rat brain tissue section MALDI analyses without the use of a mask according to the invention (A, C), or with the use of a mask according to the invention with a thickness of 65 μm and square openings with a side dimension of 500 μm (B) or 240 μm (D). The signal intensities for m/z ratios of 900 to 5500 are displayed.

FIG. 13. Rat brain tissue section MALDI analysis using of a mask according to the invention with a thickness of 100 μm and square openings with a side dimension of 100 μm, and using a MALDI-LIFT-TOF/TOF instrument (Bruker Daltonics, Bremmen, Germany) with an incidence angle of 50°. The really irradiated area corresponds to an area of about 17×75 μm². The signal intensities for m/z ratios of 600 to 3000 are displayed.

FIG. 14. SIMION 3D™ v6 simulation of electrical field lines and of ions trajectories for an electrode constituted of an opening of 240 μm with a thickness of 65 μm set at 20 kV, the last field potential being set to 19 k.

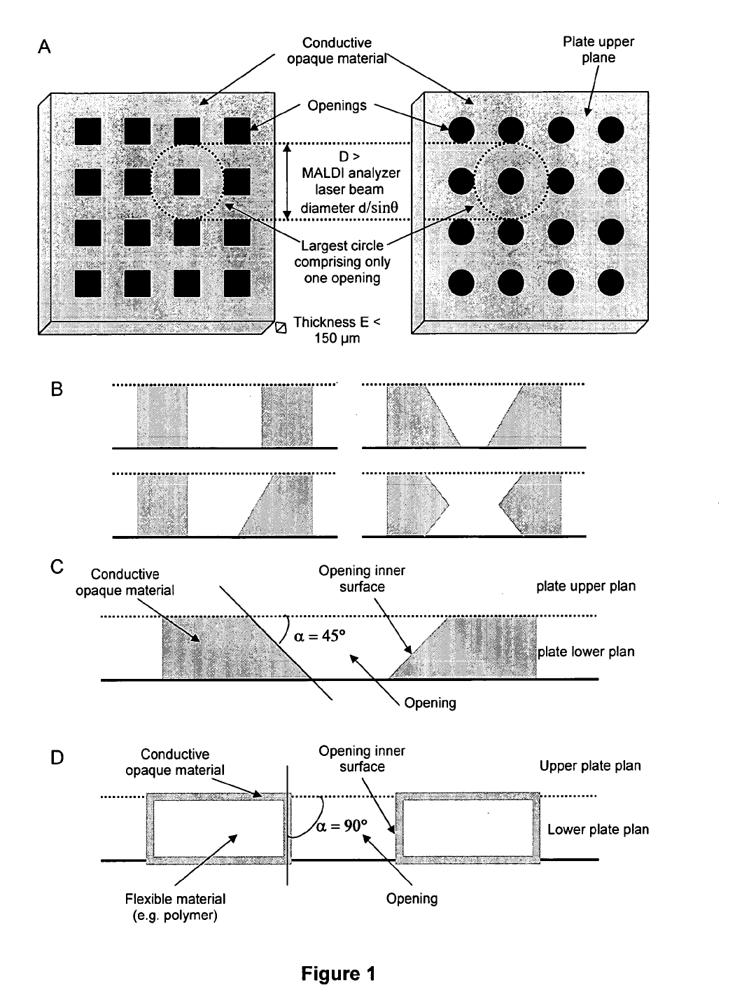

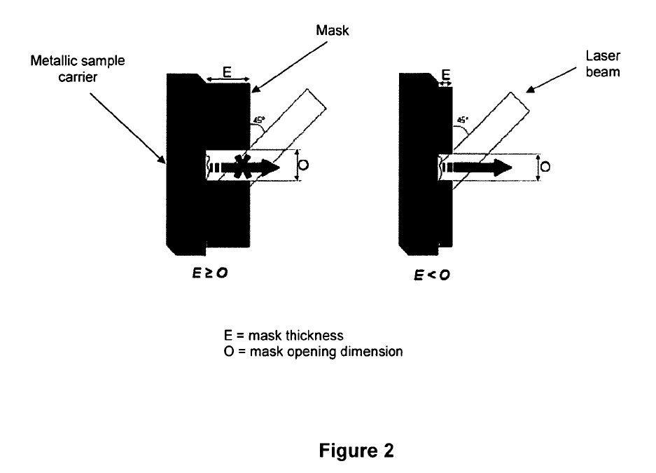

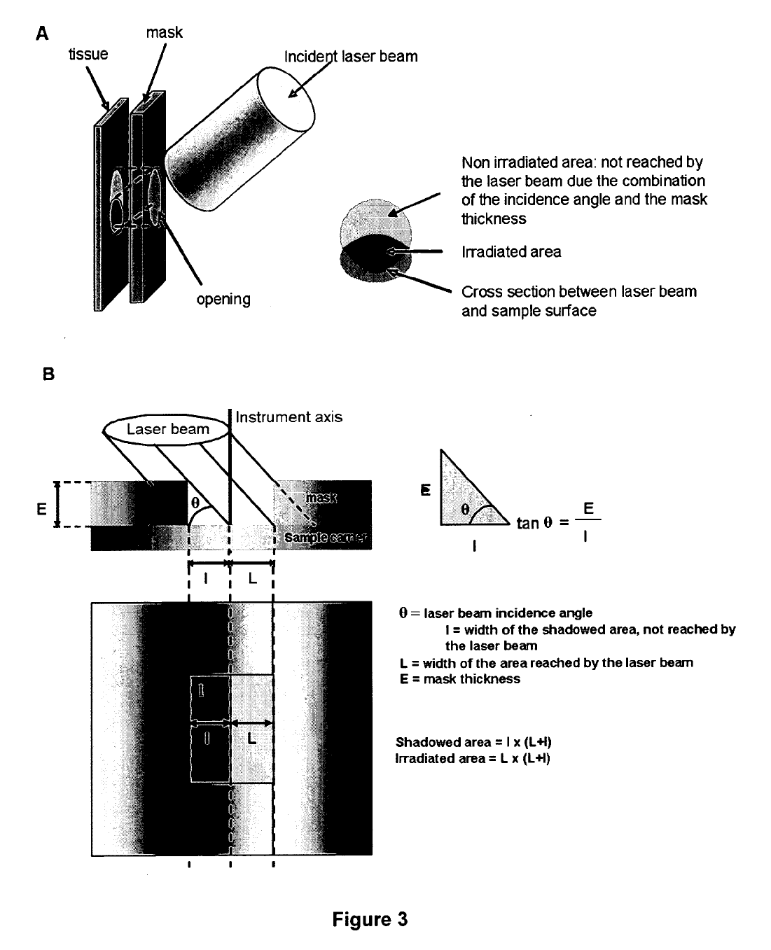

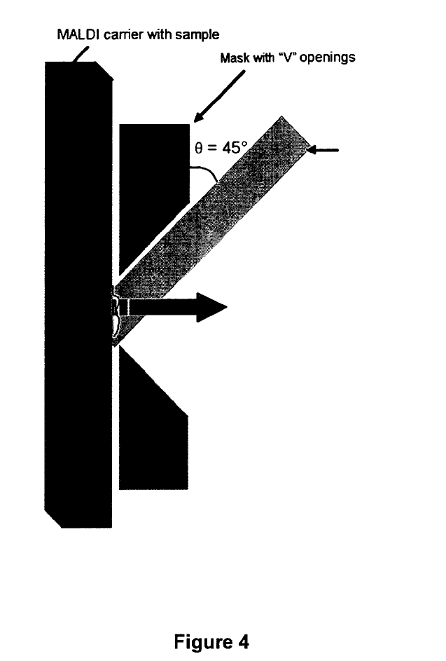

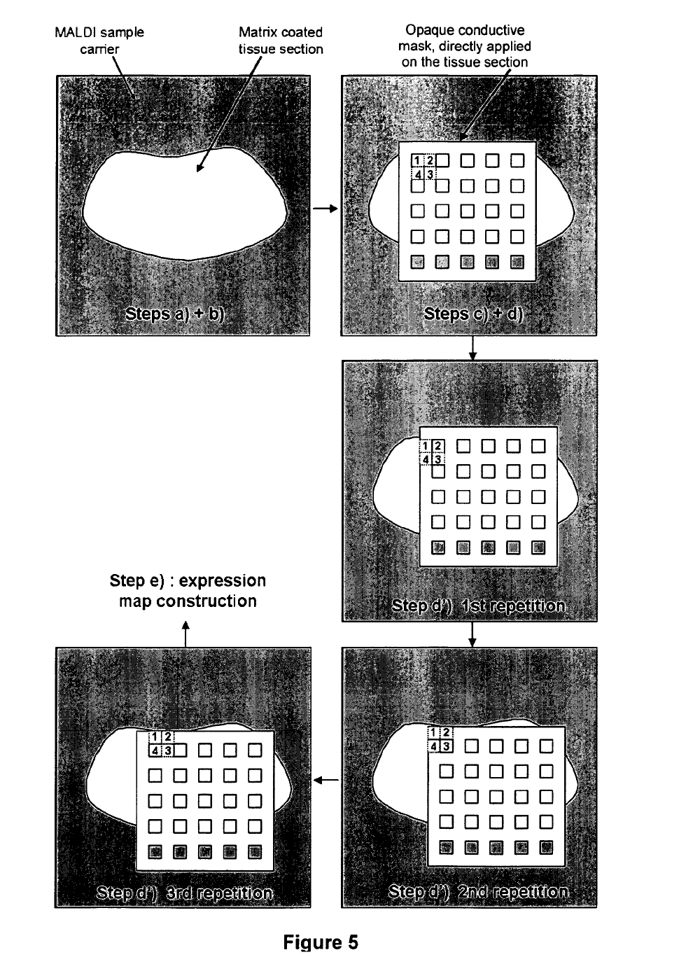

### EXAMPLES

### Example 1

### Process of Manufacture of a Mask According to the Invention

1.1 Masks with an Angle α Between the Inner Surface of the Opening and the Plate Upper Plane of About 90°

Masks with an angle α between the inner surface of the opening and the plate upper plane of about 90° made of silicon with a thickness inferior or equal to 100 μm and square openings with a side dimension d<500 μm, spaced by a distance superior to a fixed, desired distance corresponding to the laser beam radius of a MALDI analyzer for which the mask is designed, were prepared using the following process:
A silicon wafer is thinned using wet etching (like TMAH or KOH attack) until the remaining silicon wafer displays a thickness inferior or equal to 100 μm,
The resulting silicon plate is cleaned using a “piranha” cleaning solution (H₂SO₄+H₂O₂),
The cleaned silicon plate is coated with a 10 μm thick layer of negative photoresist SU-8 (Microchem),
The negative SU-8 coated silicon plate is then UV irradiated through a chromium coated glass protection having shapes of the desired configuration corresponding to the future mask openings,
The SU-8 layer is removed in areas protected by the chromium coated glass protection, which correspond to the areas of the desired openings, using a development solution (Microchem SU-8 Developer, which is constituted of 1-Methoxy-2-propyl acetate, CAS: 108-65-6),
The areas in which the unexposed SU-8 has been removed, are attacked using Inductively Coupled Plasma (ICP) by following the Bosch process (as described in DE4241045). This step results in openings of the desired configuration in the silicon plate,
Finally, the resulting mask is cleaned using the “piranha” solution.

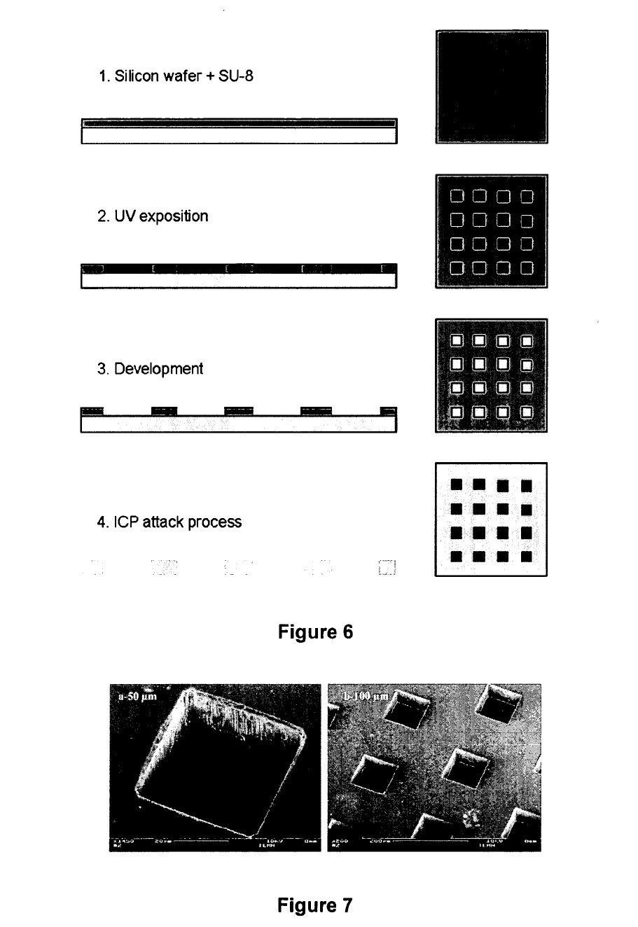

The main crucial steps of this process are presented in FIG. 6.

The masks obtained by this process have square openings with lateral surfaces perpendicular to the mask plan. Using this process, masks with a 100 μm or 65 μm thickness and openings with a side dimension of 50, 100, 240 and 500 μm have been prepared. Scanning electron microscopy (SEM) photographs of such masks are displayed in FIG. 7.
1.2 Masks with an Angle α Between the Inner Surface of the Opening and the Plate Upper Plane of About 55°

Masks with an angle α between the inner surface of the opening and the plate upper plane of about 55° made of silicon with a thickness of about 120 μm and square openings with a side dimension d of 270 μm in the upper plane and about 100 μm in the lower plane, spaced by a distance superior to a fixed, desired distance corresponding to the laser beam radius of a MALDI analyzer for which the mask is designed, were prepared using the following process:
A silicon wafer of 380 μm thickness is attacked with HF (1%) to remove native oxide,
Nitride (Si₃N₄) is deposited on both faces of the silicon wafer,
AZ 5214 photoresist (Shipley) is deposited on the upper surface (speed 3000, acceleration 1000, duration 7 seconds), and the coated wafer is cured at 120° C. during 60 seconds,
The AZ 5214 photoresist coated upper surface of the silicon plate is then UV irradiated through a chromium coated glass protection having circular openings with a diameter of 400 μm, and the plate is cured at 120° C. during 60 seconds,
The AZ 5214 photoresist coated upper surface of the silicon plate is then UV irradiated on the whole surface during 60 seconds and the AZ 5214 photoresist layer is removed in areas not protected by the chromium coated glass protection, using a pure metal ion free (MIF) developer during 20 seconds. The plate is rinsed using deionized (DI) water,
The circular openings are reported on nitride (Si₃N₄) by a Reactive Ion Etching (RIE) etching (plasma CHF3/CF4),
The photoresist is removed using “piranha” solution;
The areas corresponding to the desired openings are attacked using wet etching with TMAH (speed 0.5 μm/minute) to create the openings. Depending on the desired thickness, wet etching is performed during 200 to 480 minutes. Due to the crystalline structure of silicon, the circular areas give rise to square openings into silicon;
Both Si₃N₄ coated faces of the plate are then attacked using RIE (125W, 50 mTorr, CF4:40, CHF3:40, about 7 minutes 30 seconds);
The lower face of the plate is etched using ICP-STS to lower total thickness; and
Finally, the resulting mask is cleaned using the “piranha” solution.

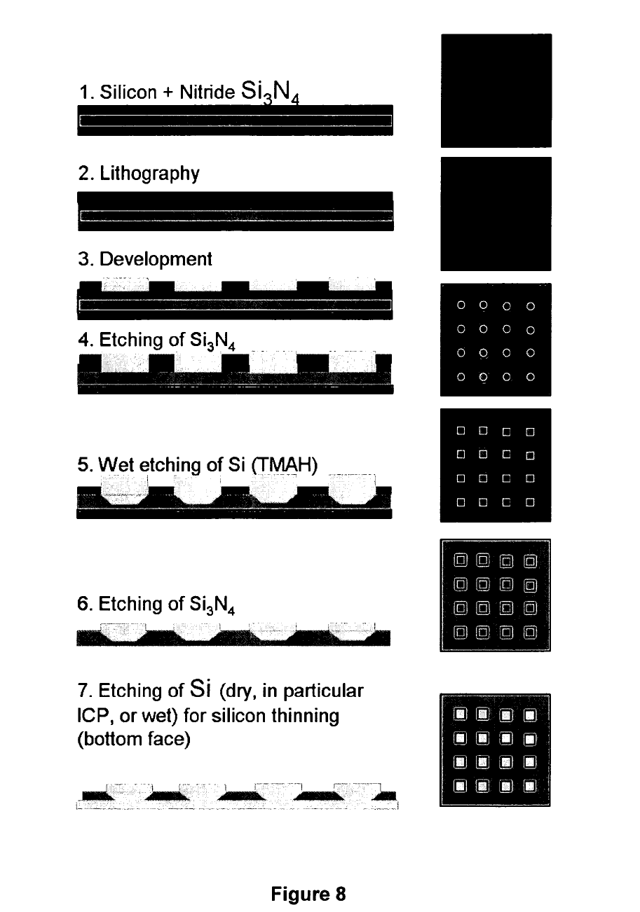

The main crucial steps of this process are presented in FIG. 8.

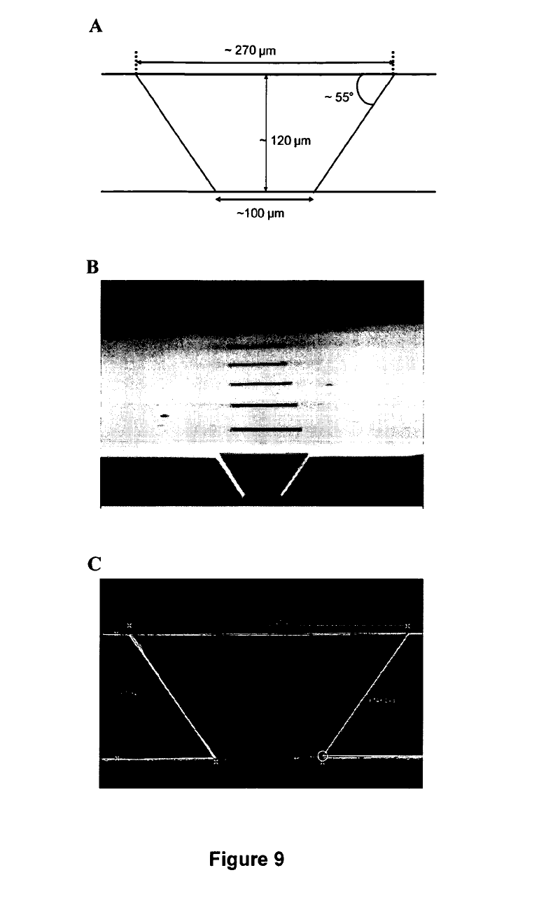

Scanning electron microscopy (SEM) photographs of such masks are displayed in FIG. 9.

Other masks of 95 μm external dimension, 36 μm internal dimension, 42 μm thickness and α=54.7° were machined from a silicon wafer using protocol described above except that the chromium coated glass protection has square openings instead of circular openings. The distance between each square of 95 μm is 5 μm.

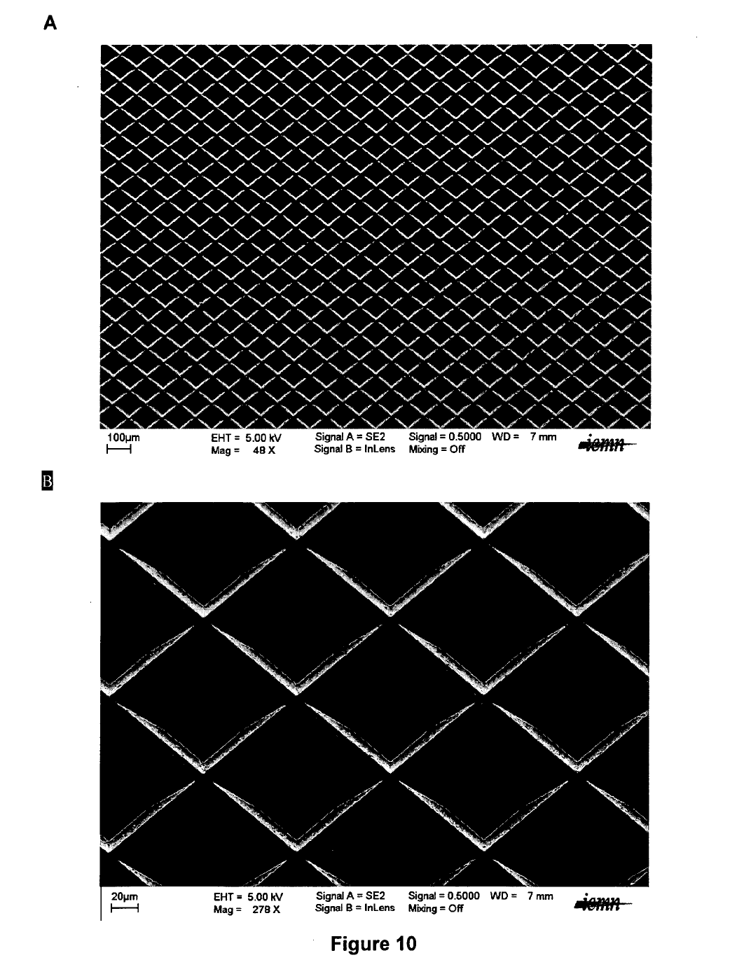

Scanning electron microscopy (SEM) photographs of such masks are displayed in FIG. 10.

### Example 2

### Use of a Mask According to the Invention for MALDI Analysis of Tissue Sections

Masks according to the invention, produced by the process described in Example 1 were tested for MALDI analysis of standard peptides mixes and rat brain tissue sections.
2.1 MALDI Analysis of Standard Peptides Mixes Using Masks According to the Invention

Masks were first tested on standard compounds (peptide mix comprising the following peptides: angiotensin II, SP-amide, ACTH(7-38), ACTH(18-39), bovine insulin, ACTH(7-39), and bovine ubiquitin) using a Voyager-DE STR MALDI time-of-flight instrument (Applied Biosystems, Framingham, Mass., USA) equipped with a 3 Hz pulsed nitrogen laser at 337 nm. Mass spectra were recorded in the linear mode using delay time of 150 ns and accelerating voltage of 25 kV. This MALDI instrument has a laser with an incidence angle of 45° and a laser beam section of 120×150 μm, corresponding to a mean laser beam diameter of 135 μm.

A MALDI sample carrier coated with the above described peptide mix and HCCA matrix was analyzed with a mask with a thickness of 65 μm and square openings having a side dimension of 500 μm or 240 μm, or a mask with a thickness of 100 μm and square openings having a side dimension of 100 μm or 50 μm.

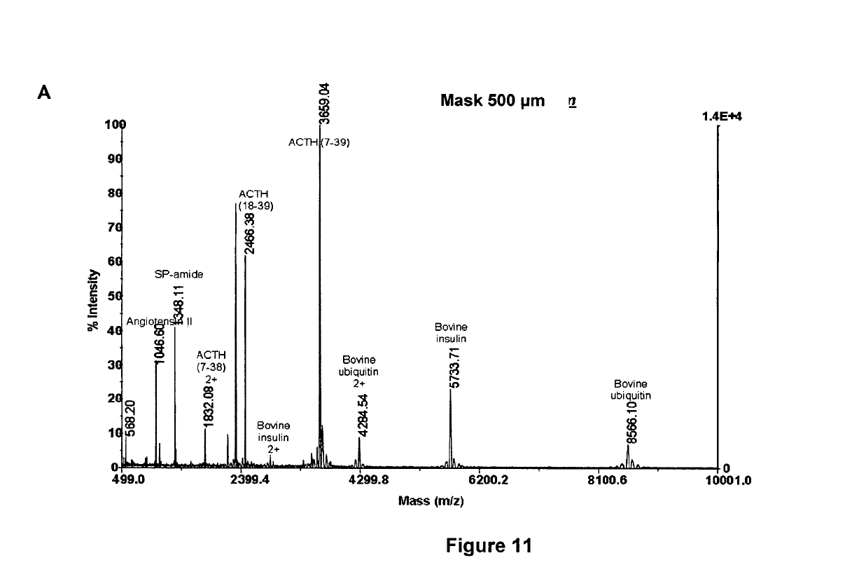

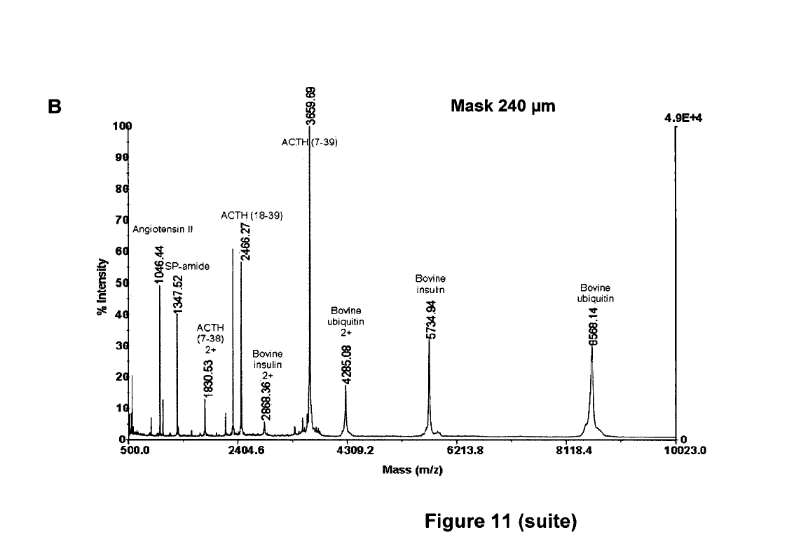

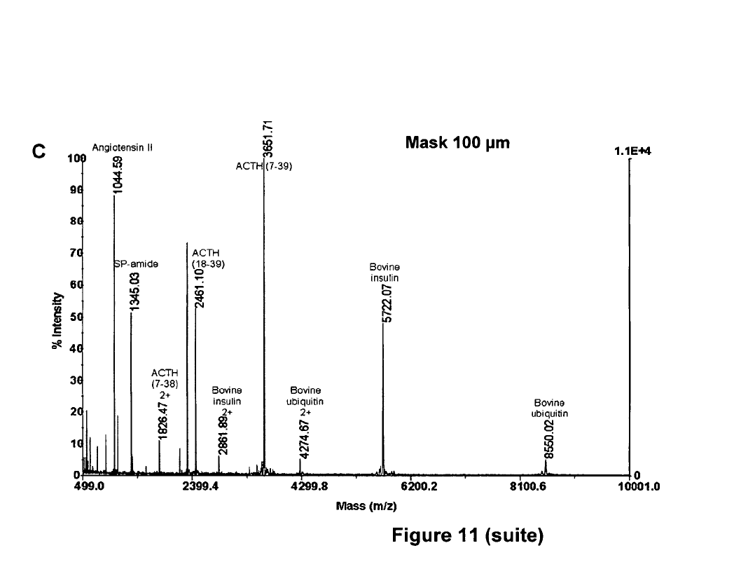

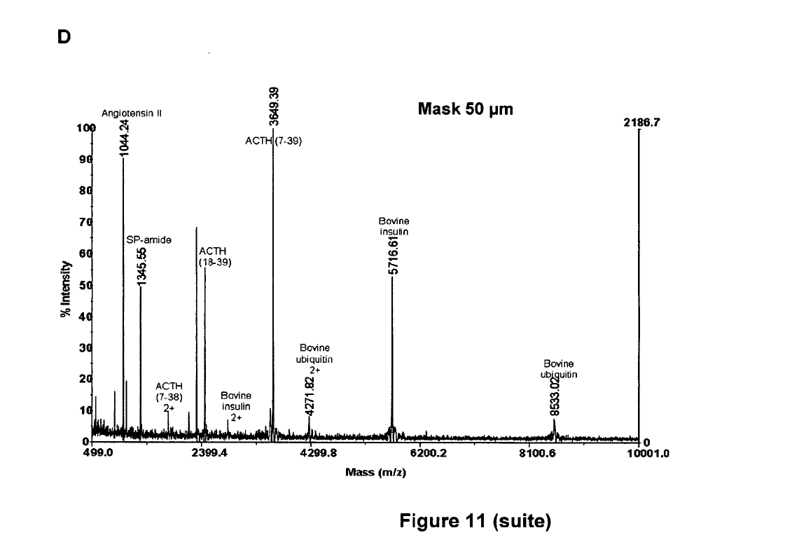

The results are displayed in FIG. 11, and show that very satisfying spectra are obtained, in particular with respect to signal intensity. Analyses performed with the mask with 50 μm openings show a signal intensity (maximum signal intensity around 2200) lower than those performed with the other masks. Signal intensities obtained with masks displaying 100 or 500 μm openings are higher and comparable (maximum signal intensity around 1.4 and 1.1 10⁴). For the mask with 240 μm openings, a significant signal intensity increase can be observed (maximum signal intensity around 4.9 10⁴).

In addition, a more precise analysis shows that the signal intensity of ions of high m/z ratios is significantly increased with masks of 240 μm (for bovine ubiquitin MH₂²⁺=4282, bovine insulin MH⁺=5716, and bovine ubiquitin MH⁺=8568) and 100 μm (for bovine insulin MH⁺=5716) openings.

Globally, for the above mentioned peptide mix, the best results were obtained with a masks of 100 μm thickness with 240 μm openings.
2.2 MALDI Analysis of Rat Brain Tissue Sections Using Masks According to the Invention

The results obtained with standards were further confirmed in direct MALDI analysis of tissue sections. Masks with a 65 μm thickness and openings of 240 and 500 μm were thus tested for MALDI analysis of rat brain tissue sections using a Voyager-DE STR MALDI time-of-flight instrument (see above).

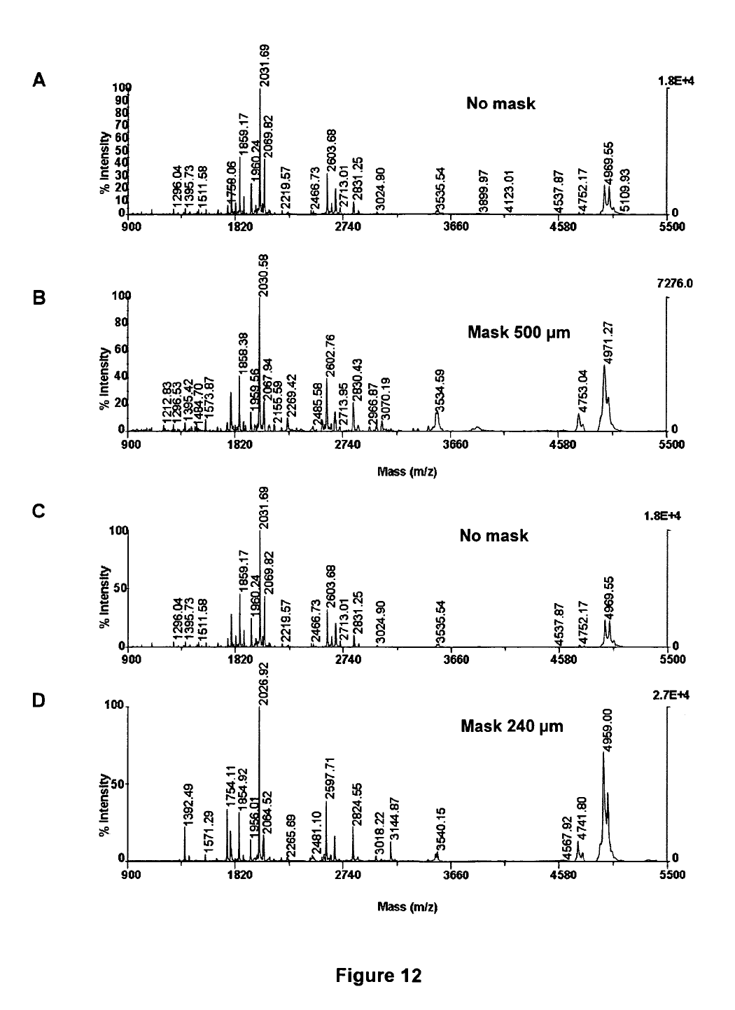

The results are displayed in FIG. 12, and show that for masks with 240 μm openings, a significant increase in signal intensity can be observed compared to a conventional analysis without using a mask (maximum signal intensity around 2.7 10⁴ compared to 1.87 10⁴).

In addition, as previously described with standards, a significant signal intensity increase of high m/z ratios (superior to 3000) is observed when using masks (240 or 500 μm openings).

Masks were also tested using a new Bruker Daltonics demonstration MALDI-LIFT-TOF/TOF analyzer (Bruker Daltonics, Bremmen, Germany), which has an incidence angle of 50°, and a laser beam diameter d of 75×75 μm². The results obtained confirmed those previously obtained with another configuration of MALDI-TOF instrument.

In particular, masks with a 100 μm thickness and 100 μm openings. The really irradiated area in this configuration corresponds to an area of 17×75 μm².

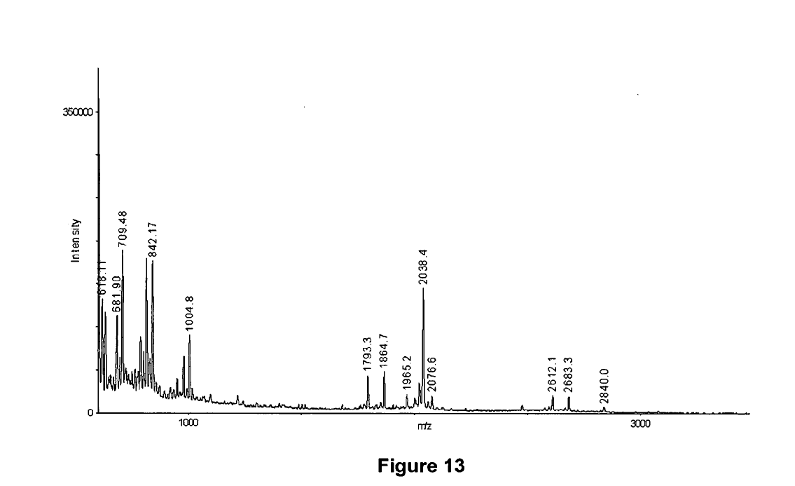

The results obtained are displayed in FIG. 13 and show that such masks allow to observe spectra with a usual signal intensity, while restraining the area analyzed to 17×75 μm². With other masks features, the irradiated areas might be reduced to about 15×50 μm?.

These results confirm the possibility to use the masks according to the invention to decrease the size of the area analyzed, and highlight their easy adaptation on any type of MALDI instrument.
2.3 Hypotheses Explaining the Observed Signal Intensity Increase

Several hypotheses can be mentioned to explain the particularly good results obtained with masks displaying 240 μm openings.

One possible explanation would be a modification of the electrical field in the 1^{st} acceleration region of the source. Indeed, the application of a conductive support comprising openings will necessary lead to a modification of the electrical field.

To study this hypothesis, simulations of the electrical field lines, as well as of the ions trajectories, have been performed using the SIMION 3D™ v6 software (available from Scientific Instrument Services, Inc. 1027 Old York Road. Ringoes, N.J. 08551, USA).

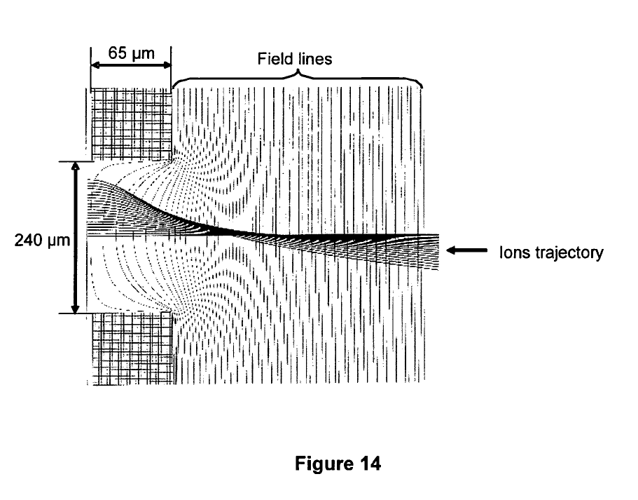

A simulation with a simple opening of 240 μm between two electrodes of 65 μm thickness on which is applied an electrical potential of 20 kV (conventional value for MALDI-TOF instruments), and in which the acceleration zone (200 mm length, here) is delimited by a flat electrode set to 19 kV, shows a significant curving of the electrical field lines around the opening, whereas electrical field lines remain straight in other regions (see FIG. 14), as usually expected for an electrical field induced by a potential difference between two flat electrodes.

In addition, the simulation of ions trajectories in this configuration shows that the ions beam is focused by such an electrical field (see FIG. 14). Such a better focusing of ions might be the reason for the observed increased sensitivity when using a mask with 240 μm openings. Such a better sensitivity, coupled to the various events accompanying the desorption/ionization process, might explain the significant increase in signal intensity observed for high m/z ratios.

### 2.4 CONCLUSIONS

The above described results clearly show that it is possible thanks to the masks according to the invention to significantly decrease the dimensions of the area irradiated by the MALDI laser without decreasing the signal intensity of the observed ions.

These results demonstrate that it is at least possible to decrease the analyzed areas down to about 15×75 μm², i.e. 1125 μm². Using other masks openings features, it might even be decreased to about 15×50=750 μm².This resolution is already better than was may be obtained without the masks, since it is not currently possible to focalise the diameter of a MALDI laser to less than about 50 μm (which corresponds to an area of about 50×50=2500 μm²) without decreasing the signal intensity.

In addition, it has even been observed that the presence of a mask according to the invention on the tissue section sample may result in a significant increase in the signal intensity, in particular for high m/z ratios. This result has been obtained both on standards peptide mixes and on rat brain tissue sections. Although the precise mechanism leading to this surprising increase in signal intensity is not completely elucidated (it might come from a better focusing of ions due to a modification of the electrical field lines by the mask), this result is particularly interesting for tissue section MALDI imaging, since most of the prior art technologies used to improve the resolution tend to result in a signal decrease.

Finally, a huge advantage of the masks according to the invention is certainly their easy adaptability on any MALDI analyzer, since their use does not involve any material modification or investment in new equipment.

### BIBLIOGRAPHY

1. Hillenkamp, F.; Dreisewerd K. 49th ASMS Conference on Mass Spectrometry and Allied Topics, Chicago, Ill., May 27-31, 2001
2. Dreisewerd K. et al, Int. J. Mass Spectrom. Ion Processes 1995, 141, 127-148
3. Caprioli, R. M. et al; Anal. Chem. 1997, 69(23), 4751-4760
4. Schwartz S A et al, J Mass Spectrom. 2003 July;38(7):699-708
5. J. Wei, J. M. Buriak and G. Siuzdak, Nature, 1999, 399, 243-246.
6. E. P. Go, J. V. Apon, G. Luo, A. Saghatelian, R. H. Daniels, V, Sahi, R. Dubrow, B. F. Cravatt, A. Vertes, and G. Siuzdak, Anal. Chem. 2005, 77, 1641-1646.
7. B. Salhi, B. Grandidier and R. Boukherroub, J. Electroceram. 16 (2006) 15-21
8. M.-J. Kang, J.-C. Pyun, J.-C. Lee, Y.-J. Choi, J.-H. Park, J.-G. Park, J.-G. Lee and H.-J. Choi, Rapid Comm. Mass Spectrom. 2005, 19, 3166-3170.
9. J. A. McLean, K. A. Stumpo, and D. H. Russell, J. Am. Chem. Soc. 127 (2005) 5304
10. A. Ulman: An Introduction to Ultrathin Organic Films: From Langmuir-Blodgett to Self-Assembled Monolayers, Academic Press, Boston, 1991.

1. A mask for use in mass spectrometry tissue section analysis, comprising a plate with an opaque external surface and having a thickness of less than 150 μm, said plate comprising regularly spaced openings, wherein in the plate upper plane, the diameter D of the largest circle comprising only one opening is superior to the diameter d of a mass spectrometer laser beam divided by sin θ, wherein θ is the mass spectrometer laser beam incidence angle with respect to the sample plane.

2. The mask of claim 1, wherein geometric form of the openings in the plate plane and the angle α between the inner surface of said openings and the plate upper plane are such that, for a laser incidence angle θ comprised between 30° and 90°, the area of sample really irradiated by the laser beam is inferior to the area of the laser beam.

3. The mask of claim 1, wherein said plate has an opaque and conductive external surface.

4. The mask of claim 3, wherein the plate material is selected among silicon, stainless steel, plastic polymers.

5. The mask of claim 4, wherein the plate is constituted of a silicon wafer.

6. The mask of claim 5, wherein at least one side of said mask is further coated with a highly conductive material.

7. The mask of claim 6, wherein all openings of said mask display identical geometric forms.

8. The mask of claim 7, wherein said openings display a rectangular or elliptic form in the plate plane.

9. The mask of claim 1, wherein the inner surface of said openings and the plate upper plane form an angle α of 30-90°.

10. The mask of claim 9, wherein the inner surface of said openings and the plate upper plane form an angle α of 30°, 45°, 50°, 60° or 90°.

11. The masks of claim 1, wherein said mask further displays a layer of porous silicon; semi-conductor nanowires arrays; gold nanoparticles arrays; or porous silicon and gold nanoparticles composite arrays on the external surface intended to be in contact with the sample.

12. A process for manufacturing the mask of any of claims claim 1, comprising:
a) providing a plate made of an opaque conductive material and having a thickness of less than 150 μm,
b) creating openings in said plate, wherein in the plate plan, the diameter D of the largest circle comprising only one opening is superior to the diameter d of a mass spectrometer laser beam divided by sin θ, wherein θ is the mass spectrometer laser beam incidence angle with respect to the sample plane.

13. The process of claim 12, wherein creating said openings comprises:
i) cleaning the plate,
ii) coating the plate with a positive or negative photoresist,
iii) irradiating the coated plate with UV through a chromium coated glass protection displaying such a configuration that the areas corresponding to the positions of the desired mask openings correspond either to chromium coated glass protected areas (in the case of a negative photoresist) or to not chromium coated glass protected areas (in the case of a positive photoresist),
iv) removing the photoresist in the areas corresponding to the desired openings using a development solution,
v) attacking the areas corresponding to the desired openings using dry etching to create the openings, and
vi) cleaning the obtained mask to remove asperities.

14. The process of claim 13, further comprising an optional step i1) between steps i) and ii) in which aluminium is deposited onto the plate.

15. The process of claim 13, wherein step v) is performed using Inductively Coupled Plasma (ICP) or wet etching.

16. The process of claim 12, wherein creating said openings comprises:
i) cleaning the plate,
ii) coating the plate with a silicon oxide or nitride,
iii) coating the plate with a positive or negative photoresist,
iv) irradiating the coated plate with UV through a chromium coated glass protection displaying such a configuration that the areas corresponding to the positions of the desired mask openings correspond either to chromium coated glass protected areas (in the case of a negative photoresist) or to not chromium coated glass protected areas (in the case of a positive photoresist),
v) removing the photoresist in the areas corresponding to the desired openings using a development solution,
vi) reporting the openings on the silicon oxide or nitride oxide by a Reactive Ion Etching (RIE) etching (plasma CHF3/CF4),
vii) attacking the areas corresponding to the desired openings using wet etching to create the openings, and
viii) cleaning the obtained mask to remove asperities.

17. The process of claim 16, further comprising an optional step after step viii), or between steps v) and vi), consisting in thinning down the plate to the thickness of the mask wished.

18. The process of claim 16 , wherein wet etching in step vii) is performed using anisotropic etchants KOH or TMAH.

19. The process of claim 12, wherein the openings are created using a micro machining technology selected from the group consisting of laser microsurgery, electrochemistry or hot embossing.

20. A process for manufacturing the mask of claim 1, comprising:
c) providing a rigid mould displaying the desired mask configuration,
d) casting a flexible material into the rigid mould to obtain a flexible mask with the desired configuration, and
e) coating the external surface of the resulting mask by a conductive material, said conductive material being also opaque if the flexible material used in step b) is not opaque.

21. The process of claim 20, wherein said flexible material is a silicone polymer or a photoresist.

22-24. (canceled)

25. A method for MALDI imaging of a tissue section, comprising:
a) Providing a tissue section sample on a MALDI sample carrier,
b) Depositing a suitable MALDI matrix onto the surface on said tissue section sample,
c) Depositing the mask of claim 1 directly onto the surface of said matrix coated tissue section sample,
d) Analyzing said tissue section sample in each mask opening using a MALDI mass spectrometer and storing all obtained spectra, and
e) Constructing the expression map of any desired compound of known m/z ratio using said stored spectra.
